(19) United States
(12) Patent Application Publication
(10) Pub. No.: US 2025/0345119 A1
(43) Pub. Date: Nov. 13, 2025

(54) COMPUTER-IMPLEMENTED METHOD FOR OPERATING AN X-RAY FACILITY, X-RAY FACILITY, COMPUTER PROGRAM, AND ELECTRONICALLY READABLE DATA CARRIER

(71) Applicant: Siemens Healthineers AG, Forchheim (DE)

(72) Inventors: Peter Fischer, Gerhardshofen (DE); Jens-Christoph Georgi, Oberasbach (DE); Marcus Pfister, Bubenreuth (DE)

(21) Appl. No.: 19/201,104

(22) Filed: May 7, 2025

(30) Foreign Application Priority Data
May 7, 2024 (DE) 10 2024 204 276.5

(51) Int. Cl. A61B 34/10; A61B 34/20

(52) U.S. Cl. CPC A61B 34/10; A61B 34/20; A61B 2034/105; A61B 2034/107; A61B 2034/2046

(57) ABSTRACT

A method for operating an X-ray facility includes supplying a vascular model of a vascular structure. A target position of an instrument that may move in the vascular structure, a starting position of the instrument in the vascular structure of the vascular model, and a path in the vascular structure of the vascular model are marked in the vascular model. Support points are defined along the path. An optimized course of projection geometries is ascertained for the support points in an optimization process of a target function using the vascular model. Apart from at least one first term, the target function also includes at least one second term that minimizes the number of changes in the projection geometry along the path due to movement of the support. Positioning parameters for actuating the positioning facility are ascertained for each projection geometry.

This application claims the benefit of German Patent Application No. DE 10 2024 204 276.5, filed on May 7, 2024, which is hereby incorporated by reference in its entirety.

### BACKGROUND

The present embodiments relate to a computer-implemented method for operating an X-ray facility with a support on which an X-ray tube assembly and an X-ray detector are arranged opposite each other, and a positioning facility for moving at least the support, in order to adjust a projection geometry. In addition, the present embodiments relate to an X-ray facility, a computer program, and an electronically readable data carrier.

For examinations and treatments on examination objects, in which a procedure is performed in a vascular structure with an instrument in a minimally invasive manner, the instrument is conventionally guided through the vascular structure to a target position for the examination and/or treatment. It is known in this connection to supply monitoring of the progression of movement using an imaging X-ray facility. For example, projection images with a low dose (e.g., fluoroscopy images) may be recorded in a specific projection geometry (e.g., in the case of C-arm X-ray facilities, characterized by the projection direction or the angulation angle of the C-arm). A large number of possibilities for preparing projection images of this kind for display on an output of the X-ray facility have already been described in this connection (e.g., via augmentation with additional items of information). Preparation may also include generating a new preparatory image based on items of information ascertained from the projection image.

For example, with a patient as the examination object, a catheter as the instrument may be guided in minimally invasive procedures through blood vessels of a section of the vascular system to a target position (e.g., a thrombus in the case of treatment following a stroke or a feeding vessel in the case of embolization of a tumor). Two-dimensional live-projection images may be used to visualize the catheter position and, for example, with administration of contrast agent, also the blood vessels.

The two-dimensional projection images are generally recorded using a specific projection geometry that may be suitably selected (e.g., by a user). With a C-arm, the projection geometry is mainly described by its angulation (e.g., the two angulation angles). The individual carrying out the procedure conventionally wants to see the instrument as well as the surrounding vessels, ideally up to the target position.

In the prior art, it is known in this regard to specify a fixed angulation or projection geometry that conventionally supplies an adequately suitable solution for carrying out the procedure as a whole. For example, the anterior-posterior direction of a patient may be selected as the projection direction.

In an improved approach, it has been proposed that a user (e.g., an individual carrying out the procedure and/or a technical assistant) manually adjusts the projection geometry in order to supply an ideal overview of the current procedural situation. However, this requires a certain amount of time, of which only a limited amount is available in minimally invasive procedures. Further, each readjustment interrupts the workflow of the intervention. Therefore, in interventions that provide such a manual adjustment, the projection geometry is frequently not adjusted even though it would enable improved visibility and improved assessment of the procedural situation. Further, collimation that may result in a dose reduction and zoom factors are also frequently inappropriately selected. In many cases, in order to adjust the projection geometry, frequent communication between an individual carrying out the procedure and a technical assistant is required.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an aid for improved image support in the case of minimally invasive procedures in vascular structures of examination objects, which aid, with optimally low stress on the users, still provides much improved image quality over the sequence of the intervention, is provided.

An embodiment of a computer-implemented method for operating an X-ray facility with a support, on which an X-ray tube assembly and an X-ray detector are arranged opposite each other, and a positioning facility for moving at least the support (e.g., a C-arm) in order to adjust a projection geometry is provided. The computer-implemented method includes supplying a vascular model, three-dimensionally describing the course of the vessel (e.g., via center lines) of a vascular structure of an examination object, from which projection images are to be recorded with the X-ray facility. A target position of an instrument that may move in the vascular structure is marked in the vascular model. The computer-implemented method includes supplying a starting position of the instrument in the vascular structure of the vascular model, supplying a path in the vascular structure of the vascular model from the starting position of the instrument to the target position, and defining support points along the path. The computer-implemented method includes ascertaining an optimized course of projection geometries for the support points in an optimization process of a target function using the vascular model, where, apart from at least one first term that is based on the optimization of the image contents for an observer in the case of a medical instrument situated at the respective support point, the target function also includes at least one second term that minimizes the number of changes in the projection geometry along the path due to movement of the support. The computer-implemented method includes ascertaining positioning parameters for actuating the positioning facility for each projection geometry.

The support may be a C-arm. C-arm X-ray facilities may be used in vascular examinations and play a supporting role in vascular treatments (e.g., in angiography systems or in some other way in operating rooms). Although many example embodiments below are based on C-arm X-ray facilities, the present embodiments may also be applied to other types of support (e.g., those with robotic arms, via which the X-ray detector and the X-ray tube assembly may be moved separately).

The examination object may be, for example, a patient, and the vascular structure may be a section (e.g., a vascular tree) of its vascular system. However, the subject matter described here is also conceivable in procedures on workpieces, and the like, which may have, for example, porous or other vascular structures.

In one embodiment, it is provided that specific ascertainable items of input information (e.g., a three-dimensional vascular model of the relevant vascular structure with known target position therein, a starting position, corresponding, for example, to the current position at the ascertainment instant, and a path from the starting position to the target position through the vessels of the vascular structure) are used in order to ultimately ascertain a course of projection geometries along the path in an optimization process. However, optimization is not solely with regard to an optimum identification of the relevant items of information for guiding the instrument in the vascular structure, but also with regard to a reduction in the changes in the projection geometry. This provides that an appropriate compromise is sought that first enables good identification of the aspects that are important for navigation, but second, allows a fluid sequence of the procedure without too many interruptions. In other words, a reduced number of projection geometries may be provided in order to avoid constant repositioning of the support and frequent mental reorientation of the individual carrying out the procedure. Since automatic ascertainment of the course of the projection geometries is possible, no interaction of the user is necessary here either. Ascertaining the positioning information may also enable automatic adjustment with a change in the projection geometry. The user is therefore relieved of strain here too.

This allows high-quality, accurate navigation of the instrument to the target position as well as a fast, optimally low-interference sequence (e.g., expediting) of the minimally invasive procedure. An efficient implementation moreover makes it possible to keep the X-ray dose low and, for example, with patients as the examination objects, to reduce the amount of contrast agent to be administered.

The at least one first term and the at least one second term may be provided with a weighting factor in the target function. The influence of the respective optimization aim may be appropriately selected via weighting factors of this kind. In example embodiments, it may be provided that the weighting factors and/or its ratio is selected as a function of a user input. Users may thus influence the optimization result according to their preferences. A user who places more value on perfect viewing angles may select a higher weighting of the at least one first term, while a user who places more value on optimally few changes in the projection geometries may place a higher weighting on the at least one second term.

The projection geometry at geometry parameters describing the respective support points may be used as optimization parameters. The optimization parameters may describe (e.g., for each support point) at least one angle of the C-arm (e.g., its angulation). In one embodiment, two angles of the C-arm are used. Further optimization parameters may describe translational positions of the C-arm if these may be influenced by the positioning facility and/or manual intervention. Beyond the pure positioning of the support, which will be discussed in more detail below, zoom and collimation may also be optimized in the optimization process. Optimization parameters may accordingly also be collimation parameters and/or zoom parameters.

Using the positioning parameter sets for the positioning facility, it is possible to actuate this facility directly in order to adjust the corresponding projection geometry. On reaching a support point at which a change exists, it is possible to directly adjust the projection geometry (e.g., after confirmation by the user). The method therefore enables optimum preparation of the subsequent course of the minimally invasive procedure.

In example embodiments, it may be provided that the vascular model to be supplied is ascertained from a three-dimensional image dataset of the examination object, which is or will be registered with a coordinate system of the X-ray facility. For example, trained analysis functions that may determine center lines and/or boundaries of the vessels may be used for ascertaining the vascular model. Analysis functions of this kind are already widely known in the prior art and may work, for example, with segmentation. The three-dimensional image dataset may be recorded with the support itself (e.g., by rotation of the recording arrangement including X-ray tube assembly and X-ray detector around the examination object; as a C-arm CT) or else may have been recorded pre-interventionally (e.g., before the interventional procedure with the instrument). The three-dimensional image dataset may be, for example, a magnetic resonance image dataset and/or a computed tomography image dataset. If the three-dimensional image dataset was not recorded with the X-ray facility or before a repositioning of the support and/or of the examination object, which may not be followed using sensors, for example, known 2D-3D or also 3D-3D registration techniques may be used to achieve registration of the three-dimensional image dataset and thus also of the vascular model with the coordinate system of the X-ray facility.

For example, the three-dimensional image dataset, possibly in conjunction with the vascular model, may also be used in a planning phase in order to manually and/or automatically select the target position there (and thus also in the vascular model), as is basically known. At least partially automatic ascertainment of the target position (e.g., via detection of a thrombus or tumor and/or, for example, in the case of an embolization, flow simulation, may also be provided.

In a development, it may be provided that the starting position as the current position of the instrument is ascertained from two-dimensional projection images (e.g., fluoroscopy images) of at least one current projection geometry (e.g., additionally using the vascular model and/or three-dimensional image dataset) and/or using a tracking system of the X-ray facility. For example, the current position of the instrument, as is basically known, may be tracked in real time in two-dimensional fluoroscopy images. If a registration does not already exist anyway, this two-dimensional representation of the instrument and the vascular structure may be registered with the three-dimensional representation of the vascular structure (e.g., the vascular model). In such a case, but also in the case of registration that has already been established, movements of the patient (e.g., cyclical movements), such as heartbeat and/or breathing, may be taken into account accordingly. In the case of the position location, the fact that the instrument is bound to the inside of the vascular structure may also be taken into account. The result is in each case a current, three-dimensional position of the instrument, which represents the starting position in the initial situation that is to be assessed for the optimization process. However, alternatively or in addition, as part of the present embodiments, it may also be provided that the X-ray facility has a tracking system in order to ascertain the current three-dimensional position of the instrument. Tracking techniques that are basically known in the prior art may be used in this case (e.g., electromagnetic tracking, opto-acoustic tracking, and the like). Finally, it is also possible to start from a planned starting position (e.g., a branch in a vascular tree) that leads to the target position, and the like. A starting position of this kind may be marked in the vascular model, like the target position.

Various approaches may be used for ascertaining the path (e.g., suitable route-finding functions that can find a route between two points in a vascular structure). For example, it may be provided that the path is determined as the shortest connection through the vascular structure from the starting position to the target position. However, constraints may also be used here (e.g., specific necessary sizes of vessels to be used), and the like. In vascular trees in the vascular system of a patient, which have not been incorrectly imaged, it is frequently the case that only a single possible route has to be found via the route-finding function.

For selecting the support points, embodiments provide that support points are defined at least at intersections of the vascular structure at which a plurality of vessels converge (e.g., at bifurcations) and/or are selected at least partially at a specific interval along at least one center line of a vessel or vascular section of the path. In one embodiment, suitable projection geometries may be ascertained at least for points at which a plurality of possibilities exist for continuation of the instrument owing to branches (e.g., for bifurcations therefore). It is precisely at bifurcations that it is extremely important to obtain a good overview of the overall situation in order to be able to proceed with the instrument accurately, gently, and completely correctly, so suitable items of information may be purposefully supplied by the method proposed here. Further support points may also be defined between intersections. These may be defined at a regular interval between bifurcations, but also using features of the vascular structure (e.g., in narrow curves). In the case of regular intervals, the intervals may be selected, for example, in the range of 1 to 5 mm.

At least one of the at least one first term may be selected from the group including: for support points at and/or in a tolerance range around bifurcations of the vascular structure, a term for minimizing the deviation of the projection direction of the projection geometry from the cross product of the running direction of the vessels of the bifurcation at the bifurcation in accordance with the vascular model; for example, for support points that are not located at a bifurcation and/or are located outside of the tolerance range, a term that minimizes the optical foreshortening of at least the vessel in which the support point is located, and/or of all vessels, visible in the projection geometry, of the path from the support point to the target position, is weighted less (e.g., as the interval from the support point increases, in the vascular structure); a term that promotes visibility of the instrument in the center of a projection image recorded with the projection geometry; a term that maximizes the visible length of vessels along the path from the support point to the target position; a term that minimizes the dose load for the examination object and/or at least one person carrying out the treatment and/or operator; and a term that minimizes the shadowing of at least part of the vascular structure (e.g., of the instrument and/or of the path from the support point to the target position) by other structures (e.g., bones) of the examination object.

At bifurcations, the deviation of the projection direction of the projection geometry from the cross product of the running direction of the vessels or vascular sections of the bifurcation is to be minimized. In other words, in the case of branches, the cross product between straight lines that are fitted to the vessels may indicate the preferred viewing direction/projection direction because the vascular sections then lie parallel to the image plane of the two-dimensional projection image and may be effectively resolved and perceived. The deviation from this optimum projection direction may be evaluated, for example, with penalty costs in the target function.

It is possible for the optical foreshortening of vessels (e.g., those that are relevant to the guiding of the instrument) to be minimized (e.g., outside of support points). This may refer to the vessel or the vascular section in which the support point is located, but may also relate to a plurality of vessels along the path or even all vascular sections along the path through to the target position. First, in this connection, the optical foreshortening may be weighted less as the interval from the support point in the vascular structure increases. Second, however, with the administration of contrast agent, the entire route through to the target position may be relevant, so the weighting along the entire path may also be selected to be the same with the administration of contrast agent.

Since users conventionally want the instrument in the center of the image, a first term may also promote visibility of the instrument in the center of a projection image recorded with the projection geometry. For example, a central region, in which the instrument is acceptable, may be defined in this connection. This may also be implemented as part of a constraint.

The visible length of vessels along the path from the support point to the target position may also be maximized by a suitable first term. This may additionally or alternatively be used to minimize the optical foreshortening. A projection geometry that best shows the entire remaining path from the support positions to the target position may therefore be sought. This may be provided, for example, for injections of contrast agent where more than the immediate surroundings of the instrument is relevant.

Further, first terms may be aimed at minimizing the dose load, and, more precisely, for the examination object, as well as for at least one individual carrying out the treatment and/or operator. Specific arrangements of the X-ray tube assembly and of the X-ray detector result in, for example, a higher dose load for the examination object and, for example, also users (e.g., therefore, an individual carrying out a procedure (individual carrying out the treatment) and/or a technical assistant (operator)). Arrangements of this kind may be avoided by appropriate first terms. Dose models, and the like, may be used in this connection, which, for example, also take scattered radiation into account, as well as dose distributions specified (e.g., in look-up tables or the like for specific projection geometries) or known dose distributions. Appropriate approaches are known, for example, from dose monitoring and may also be used as part of the optimization process.

Additionally or alternatively, a term that minimizes the shadowing of at least part of the vascular structure (e.g., of the instrument and/or of the path from the support point to the target position) by other structures (e.g., bones) of the examination object may be used. If there are structures (e.g., bones in the case of a patient) in the examination object that therefore cast strong shadows that may restrict the image quality with regard to the guiding of the instrument, the optimization process may prevent shutdowns of this kind too via a suitable first term (e.g., a positioning of the spine in the beam path to or from the instrument or the path). Items of information on the location of structures casting shadows may be obtained, for example, from the three-dimensional image dataset that has already been mentioned.

A change in the projection geometry using an alteration of at least one positioning parameter of the support (e.g., of its angulation angles) from one support point to the next may be ascertained for the second term. If, for example, two angulation angles of a C-arm are used, a change may be identified, for example, when at least one difference in the projection angles of the adjacent support points is not zero. For example, the second term may include an appropriate mathematical expression for counting such changes.

The second term may be selected to be proportional to the number of changes in the projection geometry along the path. In this way, it is possible to achieve a simple algorithmic implementation, for example, that proves the essential aspect (e.g., number of changes in the projection geometry along the path) and, for example, in accordance with it, introduces increasing penalty costs in the target function. However, embodiments may also provide that each change in the projection geometry is weighted with a magnitude of the change. Thus, for example, changes that result in a longer and/or more laborious positioning process may result in more penalty costs than those that represent, for example, only minor corrections. Thus, ultimately shorter adjustment paths may also be promoted and thus further contribute to the efficiency. The magnitude of the change may be described by a distance measurement of the projection geometries (e.g., corresponding differences from angulation angles) or else also take into account adjustment paths of the support that are actually necessary.

In one embodiment, at least one collimation parameter that describes the collimation of projection images to be recorded and/or zoom parameters that describe the zoom of projection images to be recorded may also be ascertained for each projection geometry. Further, first and/or third terms of the target function and/or constraints yet to be discussed may also refer at least partially to the collimation and/or the zoom (e.g., as the penalty terms) if at least one element of the following is not mapped: medical instrument at the support point; vascular section of the path on the path from the support point to the target position; and target position. The more remote vascular sections may result in lower penalty values than the closer vascular sections.

In one embodiment, at least one constraint may be used as the constraint term of the target function and/or as the one to be additionally checked. Such constraints are basically known for optimization problems and may refer, for example, to technical limits of the X-ray facility, objectives of the two-dimensional projection imaging that have to be met, and the like. Specifically, it may be provided, for example, that at least one of the at least one constraints is selected from the group including: a specified arrangement of the X-ray detector above the examination object; a collision-free nature of the movement path between two projection geometries of a change; a visibility of the path to the target position for each projection geometry; and at least one restriction given by the embodiment of the X-ray facility.

An arrangement of the X-ray detector above the examination object provides that the X-ray tube assembly is arranged below the examination object and thus, for example, below an object table (e.g., patient table) of the X-ray facility, so a reduction in the exposure to radiation of users may also be achieved in this way. A collision-free nature of the movement path between two projection geometries may be checked, for example, by an anti-collision system of the X-ray facility. For example, the collision-free nature may be queried by the anti-collision system. In this way, it is possible to provide that the change may also be executed. If a user (e.g., the individual carrying out the procedure) wants to see specific aspects in each projection image, for example, apart from the instrument, the entire path through to the target position, a suitable constraint may likewise be defined. Finally, constraints may, as already mentioned, also take into account technical limits of the X-ray facility itself. It should be noted in general that constraints may also be integrated in the target function as the penalty term.

To summarize, a high-dimensional, non-convex optimization problem with constraints may therefore exist, which may be solved by a suitable solution algorithm for finding the optimum course of projection geometries via the support points. In an example embodiment, it may be provided that a Viterbi algorithm and/or a forward-backward algorithm is used for carrying out the optimization process. Solution algorithms of this kind have the advantage of finding a global optimum, but work with discrete values for the optimization parameters. In one example, with regard to the angulation angle, it is possible to work with basic values for the optimization parameters at an interval of, for example, 1° to 10°. However, other solution algorithms may also be used, as are basically known in the prior art (e.g., gradient-based methods).

In order to easily use the ascertained items of positioning information to implement image monitoring, it may be provided that a current position of the instrument in the vascular structure is tracked, where on reaching a support point, at which a change in the projection geometry takes place in accordance with the optimized course (e.g., after confirmation by a user), the positioning facility is actuated by the positioning parameter set of the projection geometry that is to be readjusted.

It should also be noted at this point that the method of the present embodiments relates solely to the operation of the X-ray facility and the adjustment thereof for recording projection images. The method includes neither the procedure as such nor the administering of contrast agents, if provided.

For example, as already stated, a current position of the instrument may be constantly tracked by evaluation of the two-dimensional projection images (e.g., fluoroscopy images) and/or by the tracking system. It is therefore possible to identify when support points are reached, and therefore, also when a change in the projection geometry should take place in the optimum course. It is then always possible for the new projection geometry to be proposed to the user. If the user activates this geometry, the user accepts the new adjustments; therefore, the new projection geometry may be automatically adjusted using the positioning information. Two-dimensional projection images eminently suitable for monitoring the guiding of the instrument may be recorded as required in this geometry.

In a development in this connection, it may be provided that the confirmation by the user takes place together with the activation of X-ray radiation and/or on operation of a foot pedal. In this way, the user does not need to use any further operating devices than those that he uses anyway to activate the X-ray radiation and to record projection images, and which, since it is foot-operated, causes little diversion from the examination object. In one embodiment, the new projection geometry may be accepted with an operation, and the recording of at least one two-dimensional projection image in this geometry may be activated. The number of interactions with the X-ray facility is thus also reduced.

A development may also provide that with a deviation by the user from the projection geometries of the optimized course, a new ascertainment takes place in accordance with the optimization process, starting from the adjusted, deviating projection geometry. This provides that when the user makes a different adjustment to the support than recommended in accordance with the optimized course, it is possible to ascertain a new optimized course once again (e.g., in real time, starting from the projection geometry selected by the user). Deviating adjustments and wishes of the user may thus be taken into account, and outstanding assistance may still be supplied in the further course of the method.

Apart from the method, the present embodiments also relate to an X-ray facility having a support on which an X-ray tube assembly and an X-ray detector are arranged opposite each another, a positioning facility for moving at least the support in order to adjust a projection geometry, and a control facility. The control facility has a first interface for supplying a vascular model, three-dimensionally describing the course of the vessel (e.g., by center lines) of a vascular structure of an examination object, from which projection images are to be recorded with the X-ray facility. A target position of an instrument that may move in the vascular structure is marked in the vascular model. The control facility also has a second interface for supplying a starting position of the instrument in the vascular structure of the vascular model, a third interface for supplying a path in the vascular structure of the vascular model from the starting position of the instrument to the target position, a defining unit for defining support points along the path, and an ascertainment unit for ascertaining an optimized course of projection geometries for the support points in an optimization process of a target function using the vascular model. Apart from at least one first term that is based on the optimization of the image contents for an observer in the case of a medical instrument situated at the respective support point, the target function also includes at least one second term that minimizes the number of changes in the projection geometry along the path due to movement of the support, and for ascertaining positioning parameters for actuating the positioning facility for each projection geometry.

All statements with respect to the method of the present embodiments may be transferred analogously to the X-ray facility of the present embodiments, and vice versa, so the advantages already identified may also be obtained with this facility. The control facility may have at least one processor and at least one storage device. Functional units of the control facility may be formed by hardware and/or software.

The interfaces may at least partially also be internal interfaces. The control facility may thus also have, for example, a tracking unit for ascertaining the current position respectively of the instrument (e.g., using two-dimensional projection images of the X-ray facility and/or a tracking system of the X-ray facility). The tracking unit may supply the ascertainment unit, but also other functional units of the control facility, with this current position via the second interface as the starting position. In addition or alternatively, the control facility may also have a path-determining unit that may obtain (e.g., likewise via the second interface) the starting position and, via the first interface, the vascular model, in order to ascertain the path through the vascular structure from the starting position of the instrument to the target position, and supply it via the third interface. As is basically known, the control facility may also have a recording unit that has the recording operation of the X-ray facility (e.g., for recording individual two-dimensional projection images, such as fluoroscopy images, and/or a projection image set for a three-dimensional image dataset). For this, the control facility may also have a corresponding reconstruction unit for reconstruction of the three-dimensional image dataset from the projection image set.

However, the control facility also has a monitoring unit for checking whether the tracked, current position of the instrument on the path reaches a support point for which the optimized course shows a change in the projection geometry, and a control unit for actuating, on reaching such a support point (e.g., after confirmation by a user), the positioning facility using the positioning parameter set of the projection geometry that is to be readjusted. The monitoring unit may use the current position of the tracking unit, which is supplied via the second interface. The monitoring unit may also be configured to monitor whether a deviation by the user from the projection geometries exists along the optimized course, and in this case, initiate a new ascertainment in accordance with the optimization process, starting from the adjusted, deviating projection geometry.

The X-ray facility may also have operating device and an output device. For example, the operating device may include a foot pedal, by which recording of two-dimensional projection images (e.g., fluoroscopy image) may be triggered. Such a foot pedal proves to be useful, for example, with minimally invasive procedures on the examination object in which a user guides the instrument in the examination object. The foot pedal may also be used to confirm the adjustment of a new projection geometry. Generally speaking, projection images recorded in the projection geometries of the optimized course (e.g., fluoroscopy images) may be output at an output (e.g., a monitor). In this connection, the basically known preparations may be provided (e.g., overlays with a three-dimensional preoperative image dataset and/or the vascular model, additionally annotations, highlighting or substitutions, such as of the detected instrument, and the like). In one embodiment, the control facility may include an output unit for the preparation and output of projection images recorded in the projection geometries along the optimized course. The output unit may be configured to highlight the path (e.g., remaining path) to the target position. Changes, pending along the path, in the projection geometry may likewise be displayed in this connection (e.g., by a change in the color of the highlighting, a marker along the path, and the like).

In one embodiment, a computer program may be loaded directly into a storage device of a control facility of an X-ray facility and has program means that, when the computer program is executed on the control facility, prompt the control facility to carry out acts of a method of the present embodiments. The computer program may be stored on an electronically readable data carrier of the present embodiments that therefore includes items of control information stored thereon. The items of control information are configured, such that when the data carrier is used in a control facility of an X-ray facility, the control facility is configured to carry out the method of the present embodiments. The electronically readable data carrier may be a non-transient data carrier (e.g., a CD-ROM).

### BRIEF DESCRIPTION OF THE DRAWINGS

FIG. 1 shows a flowchart of an example embodiment of a method;

FIG. 2 shows, by way of example, an outline of a vascular structure with support points;

FIG. 3 schematically shows a prepared projection image;

FIG. 4 shows a schematic outline of an embodiment of an X-ray facility; and

FIG. 5 shows the functional construction of a control facility of the X-ray facility.

### DETAILED DESCRIPTION

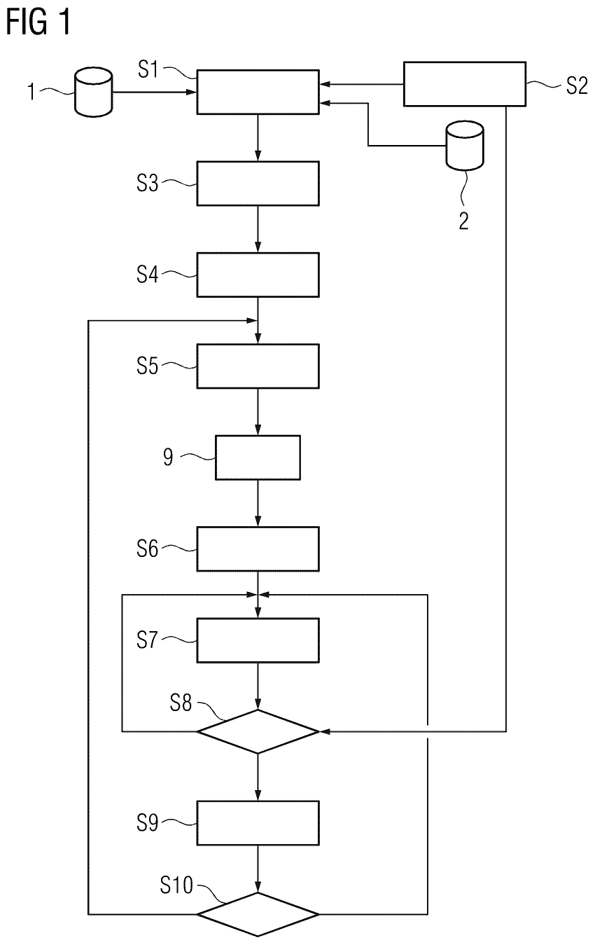

FIG. 1 shows a flowchart of an example embodiment of a method. This is used for planning image monitoring in the case of a minimally invasive procedure with an instrument (e.g., a catheter) in a vascular structure (e.g., a section of a vascular system of a patient as the examination object), and in order to adjust an X-ray facility for recording two-dimensional projection images (e.g., fluoroscopy images) for image monitoring. The X-ray facility has, as the support for a recording arrangement including an X-ray tube assembly and an X-ray detector, a C-arm that may be rotated in the present case about two axes by a positioning facility in order to adjust different projection geometries (e.g., angulations of the C-arm). Each angulation is characterized by two angulation angles. Embodiments are also conceivable in which further or other adjustment options of the support are supplied by the positioning facility. The X-ray facility also has a collimation facility that is associated with the X-ray tube assembly, and a zoom facility that is associated with the X-ray detector. These may also be adjusted by the positioning facility.

The following acts are carried out by a control facility of the X-ray facility. In act S1, fundamental items of input information are received via a first interface and a second interface. A vascular model 1 is received via a first, in the present case external, interface. The vascular model 1 may be, for example, a planning result. The vascular model 1 may be derived by at least one trained analysis function from a three-dimensional image dataset that was recorded before or at the beginning of the procedure (e.g., by detection of the center lines of the vascular structure, which may also describe the course of the vessels or vascular sections in the vascular model 1). The three-dimensional image dataset may have been recorded in this connection with a different imaging facility (e.g., as the magnetic resonance image dataset and/or computed tomography image dataset). However, the three-dimensional image dataset may also be recorded with the X-ray facility itself (e.g., by rotation of the C-arm about the examination object (C-arm CT)). In the latter case, a registration with the coordinate system of the X-ray facility for the vascular model 1 is already given; otherwise, a 2D-3D registration or 3D-3D registration may take place in an image-based manner in the case a C-arm CT, so the vascular model 1 is ideally known in the coordinate system of the X-ray facility.

A target position for the instrument has also already been marked in the vascular model 1. In the case of a minimally invasive procedure on a patient, the target position may be, for example, a thrombus to be removed or a feeding vessel for an embolization.

Further, in act S1, a starting position of the instrument in the vascular structure is received via a second interface, with a plurality of possibilities existing. First, the current position of the instrument may be tracked in act S2 anyway. Two variants are conceivable in the X-ray facility in this connection, which may also be used supplementarily. In one variant, it is possible to track the instrument in real time in two-dimensional projection images (e.g., fluoroscopy images), in that the two-dimensional representation of the instrument in the fluoroscopy images is or will be registered with the three-dimensional representation of the vascular structure (e.g., the vascular model 1). This may exploit the fact that the instrument has to moved inside the vascular structure. Respiratory and cardiac movements as well as potential deformation of the vessels owing to the instrument itself may be taken into account accordingly in this connection. Therefore, the three-dimensional position of the instrument (e.g., the tip of the instrument, such as the catheter tip) is then known. In another variant, a tracking system of the X-ray facility is used to track the three-dimensional position of the instrument. For example, electromagnetic and/or opto-acoustic concepts may be used. In this first possibility, the current position of the instrument may therefore be used as the starting position 2. In this case, the second interface is therefore internal.

However, it is also possible to specify the starting position 2 in a different way (e.g., if the instrument has not yet been introduced into the examination object). Then, for example, a planned starting point in the examination object that may be reached easily, may be used as the starting position 2. Such a starting position may also be indicated by a user (e.g., in a planning phase).

In act S3, the vascular model 1 with the target position and the starting position 2 are used (e.g., using a route-finding algorithm) to ascertain a path through the vascular structure from the starting position 2 to the target position. This may be the shortest possible path in this case. The path is supplied via a third, in the present example internal, interface.

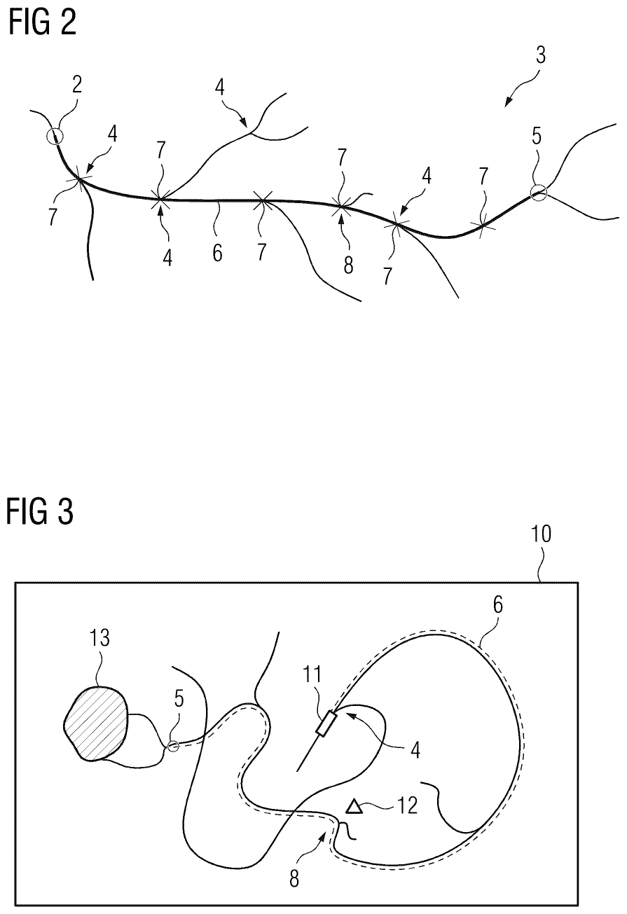

In this connection, FIG. 2 shows purely schematically a vascular structure 3 (e.g., in the present case, a vascular tree as the section of a vascular system of a patient). The vascular structure 3 includes a large number of vessels or vascular sections that are connected at bifurcations 4 (e.g., branches/intersections) and are represented by their center lines. Also shown are the starting position 2 and the target position 5 (e.g., by way of example, as the feeding vessel to a tumor, which is to be the subject of an embolization treatment). Shown in thicker lines is the path 6 from the starting position 2 to the target position 5.

In act S4, support points 7 (cf., FIG. 2) are defined along the path 6, with the starting position 2 and the target position 5 may also being used as the support points 7. In each case, support points 7 at bifurcations 4 are selected, as shown in FIG. 2. Further support points 7 may also be used (e.g., at regular intervals between bifurcations 4, as is shown in the present case, by way of example, only between the last bifurcation 4 and the target position 5).

In act S5, cf. FIG. 1 again, an optimization process then takes place to ascertain an optimized course 9 of projection geometries along the support points 7 and thus of the path 6. A specific (e.g., adjusted) projection geometry may be assumed in this connection, or else (e.g., in the case of advance planning), a completely free selection may be made. The target function for the optimization process, which in the present case takes place using the Viterbi algorithm, includes not just first terms that relate to optimum identification of relevant features in two-dimensional projection images of the projection geometry, but also at least one second term that promotes an optimally low number of changes in the projection geometry along the path 6. The first terms and the second terms may be weighted to stress respective aspects, possibly also selected by a user. In the present specific example, the angulation angles already mentioned are used as optimization parameters, with it being possible to also use further or other positioning parameters of the support (e.g., of the C-arm). Further, collimation parameters relating to the collimation may also be used, as well as a zoom parameter relating to the zoom having optimization aspects that may be included in first terms but may also be mapped by third terms. Finally, constraints are also used, so a high-dimensional, non-convex optimization problem with constraints is produced. Schematically, the optimization problem may be written down such that the sought solution L (e.g., the optimized course; described by optimization parameters for each support point 7) is

L=argmin(first terms+second term) s.t. constraints.

The target function (e.g., first terms+second term) may also include third terms as well as constraints expressed as the constraint term.

The first terms are selected from the group including: for support points 7 at and/or in a tolerance range around bifurcations 4 of the vascular structure 3, a term for minimizing the deviation of the projection direction of the projection geometry from the cross product of the running direction of the vessels of the bifurcation 4 at the bifurcation in accordance with the vascular model 1; for example, for support points 7 that are not located at a bifurcation 4 and/or are located outside of the tolerance range, a term that minimizes the optical foreshortening of at least the vessel in which the support point 7 is located, and/or of all vessels, visible in the projection geometry, of the path 6 from the support point 7 to the target position 5, is weighted less (e.g., as the interval from the support point 7 increases) in the vascular structure 3; a term that promotes visibility of the instrument 11 in the center of a projection image recorded with the projection geometry; a term that maximizes the visible length of vessels along the path 6 from the support point 7 to the target position 5; a term that minimizes the dose load for the examination object 22 and/or at least one individual carrying out the treatment and/or operator; and a term that minimizes the shadowing of at least part of the vascular structure 3 (e.g., of the instrument 11 and/or of the path 6 from the support point 7 to the target position 5) by other structures (e.g., bones) of the examination object.

It should also be noted at this point that, for example, in the case of a planned delivery of a contrast agent, the terms regarding the optical foreshortening or the visible length may be selected such that the entire remaining path 6 to the target position 5 is best represented.

The second term may be selected to be proportional to the number of changes in the projection geometry along the path 6 due to movement of the support (e.g., C-arm). In this connection, in example embodiments, each change in the projection geometry may be weighted with a magnitude of the change. Changes in the projection geometry may be established in this connection in that the difference between identical optimization parameters (e.g., angulation angles) at adjacent support points 7 is not zero.

With regard to the collimation parameter and/or the zoom parameter, a selection may be made, for example, such that, as accurately as possible, all features or image components that are not relevant to the guiding of the instrument are removed (e.g., everything apart from the instrument (that is received at the respective support point 7), the target position 5 and the vessels between the two).

Constraints may relate, for example, to a specified arrangement of the X-ray detector above the examination object, a required collision-free nature of the movement path between two projection geometries of a change, the visibility of the path 6 to the target position 5 for each projection geometry, and/or at least one limit given by the technical embodiment of the X-ray facility.

In the situation in FIG. 2 (e.g., up to the bifurcation region 8 in which a vessel branches downwards), all courses of vessels and bifurcations 4 may still be mapped in sufficiently high quality by a specific, fixed projection geometry, which, however, is to be changed at the bifurcation region 8 in order, when possible, to also represent this local bifurcation 4, such that the vessels run at least approximately parallel to the image plane of a recorded two-dimensional projection image. This necessary change in the projection geometry may be included accordingly in the optimized course 9 (cf., FIG. 1), which is the result of the optimization process in act S5. For each of the projection geometries to be used, corresponding items of positioning information for the positioning facility are also supplied in act S5 in order to then be able to actuate it immediately.

In the present example, a global optimum may be found owing to the use of the Viterbi algorithm, with discrete values being used for the optimization parameters, however (e.g., angles in the interval from 1° to 10°). Other solving methods may also be used.

In the further course or when carrying out the procedure, the first projection geometry is then automatically adjusted, if necessary, in act S6. Two-dimensional projection images (e.g., fluoroscopy images) may then be recorded in this projection geometry in act S7, possibly at the request of a user using a foot pedal of the X-ray facility. In this connection, in accordance with act S8, using the current position of the instrument supplied by the tracking act S2, it is constantly checked whether a support point 7 has been reached at which the optimized course 9 shows a change in the projection geometry. If this is the case, the projection geometry that is to be newly adjusted is proposed to the user, after which he may accept the proposed projection geometry, likewise with the foot pedal already mentioned or a different operating device, and may thus activate the automatic adjustment in act S9 in accordance with the corresponding positioning information. In one embodiment, both the automatic adjustment of the new projection geometry as well as the recording of further two-dimensional projection images (e.g., the fluoroscopy therefore) may be activated by the foot pedal.

However, since it is also possible for a user to purposefully deviate from the proposed projection geometry, it is possible to check this in act S10. In the event of a deviation, a new optimization process is started in act S5, with the starting position 2 now being the current position of the instrument and the starting point being the projection geometry adjusted by the user.

If it is established in act S8 that a change in the projection geometry is not necessary, or it is established in act S10 that the user has not changed the projection geometry, the method returns to act S7, and therefore, the recording of two-dimensional projection images, possibly on a corresponding request.

The projection images recorded in act S7 are displayed in an act that is not shown for the sake of clarity, on an output device (e.g., a monitor of the X-ray facility). The two-dimensional projection images may still be prepared, for example, by highlighting the instrument and/or the (e.g., remaining) path 6. During this preparation, changes in the projection geometry that are already pending along the path 6 may also be pointed out to the user.

FIG. 3 shows, by way of example, a prepared projection image 10. In FIG. 3, the instrument 11 (e.g., more precisely, the instrument tip) is shown highlighted, with the instrument 11 being situated exactly at a bifurcation 4 that, owing to the optimized course 9 of the projection geometries, may be viewed such that the corresponding vessels deviate in their course at least only slightly from a parallelism with the image plane. The further path 6 through to the target position 5 is also highlighted. A symbol 12 indicates that a change in the projection geometry is pending in a bifurcation region 8. The tumor 13 that is to be treated in the example may also be seen.

The instrument 11 is arranged as centrally as possible, as desired by many users, and is promoted by an appropriate first term and/or a suitable constraint.

The method is ended when the target position 5 is reached.

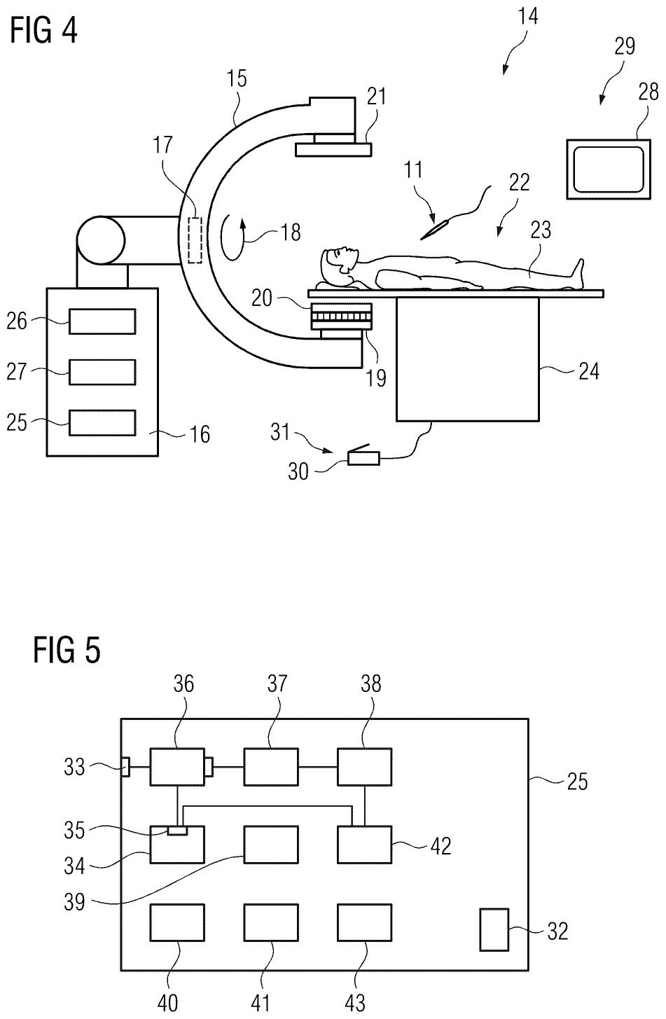

FIG. 4 shows a schematic outline of an embodiment of an X-ray facility 14. This has a C-arm 15 as the support, which is secured to a stand 16 and may be brought into different angulations via a positioning facility 17, as is schematically indicated by the arrow 18. Arranged opposite each other on the C-arm 15 are an X-ray tube assembly 19 with a collimator 20, which may likewise be adjusted via the positioning facility 17, and an X-ray detector 21. An examination object 22 (e.g., a patient 23) may be supported on a patient table 24.

Apart from a control facility 25 for controlling operation of the X-ray facility 14, in the present case, the X-ray facility also includes an anti-collision system 26 for preventing the collision of components of the X-ray facility 14 with other components, individuals, and/or further objects, as is basically known in the prior art. The anti-collision system 26 may deliver, for example, the items of information as to whether a change between projection geometries is possible without collisions. In addition, a tracking system 27 is provided for the instrument 11, which is only suggested here, and this determines the position of the instrument and may work, for example, electromagnetically or opto-acoustically.

A monitor 28 may be used as the output device 29 in order to display prepared projection images 10 (e.g., fluoroscopy images) that may be seen clearly from the site of the procedure on the patient 23. Simple user inputs may be executed by a foot pedal 30 as the operating device 31 (e.g., in order to activate radiation for recording projection images and/or for confirmation of a proposed projection geometry).

Of course, the X-ray facility 14 may also have further components (e.g., sensors) for dose monitoring and further operating devices and/or output devices.

FIG. 5 shows the functional structure of the control facility 25 in more detail with regard to some functional units. Apart from a storage device 32, the control facility 25 first includes a first interface 33 that is an external interface in the present case. The vascular model 1 may be received via the first interface as part of act S1. In accordance with act S2, a tracking unit 34 uses recorded two-dimensional projection images and/or the tracking system 27 in order to supply a current position of the instrument 11 (e.g., of its tip) via a second (e.g., internal) interface 35 (e.g., again, as part of act S1).

A path-determining unit 36 uses the current position as the starting position 2, as well as the vascular model 1 with the target position 5, to ascertain the path 6 from the starting position 2 to the target position 5 in accordance with act S3. The path is supplied by a third, again internal here, interface 44. In accordance with act S4, the support points 7 are then defined in a defining unit 37. The optimization process in accordance with act S5 may then take place in an ascertainment unit 38 (e.g., together with items of positioning information corresponding to the ascertainment).

A control unit 39 uses the items of positioning information to adjust projection geometries of the optimized course 9 in accordance with acts S6 and S9. Projection images may be recorded by a recording unit 40, as basically known (e.g., also in act S7). In the case of three-dimensional recordings (C-arm CT), a reconstruction unit 41 may be used for reconstruction of three-dimensional image datasets from projection images.

If the instrument 11 is situated along the path 6 at a different current position, supplied by the tracking unit 34, a monitoring unit 42 may monitor, in accordance with act S8, whether, in accordance with the optimized course 9, a change in the projection geometry is pending and trigger the corresponding measures. The monitoring unit 42 may also monitor, in accordance with act S10, whether the user deviates from the projection geometries of the course 9.

Finally, the control facility 25 may also have an output unit 43 in which prepared projection images 10 may be ascertained (e.g., using additional marking of positions for the change in projection geometries).

The control facility 25 may also have further functional units (not shown here for the sake of clarity) (e.g., a user interaction unit with regard to the foot pedal 30 or other operating device 31, a dose-monitoring unit for supplying items of information with regard to corresponding first terms of the target function, and the like).

In this patent application, independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for operating an X-ray facility with a support on which an X-ray tube assembly and an X-ray detector are arranged opposite each other, and a positioning facility for moving at least the support, such that a projection geometry is adjusted, the method being computer-implemented and comprising:
supplying a vascular model, three-dimensionally describing a course of the vessel, of a vascular structure of an examination object, from which projection images are to be recorded with the X-ray facility, wherein a target position of an instrument that is movable in the vascular structure is marked in the vascular model;
supplying a starting position of the instrument in the vascular structure of the vascular model;
supplying a path in the vascular structure of the vascular model from the starting position of the instrument to the target position;
defining support points along the path;
ascertaining an optimized course of projection geometries for the support points in an optimization process of a target function using the vascular model, wherein, apart from at least one first term that is based on the optimization of the image contents for an observer in the case of an instrument situated at the respective support point, the target function also comprises at least one second term that minimizes a number of changes in the projection geometry along the path due to movement of the support; and
ascertaining positioning parameters for actuating the positioning facility for each projection geometry.

2. The method of claim 1, wherein the vascular model to be supplied is ascertained from a three-dimensional image dataset of the examination object, which is or will be registered with a coordinate system of the X-ray facility.

3. The method of claim 1, wherein the starting position as the current position of the instrument is ascertained from two-dimensional projection images of at least one current projection geometry.

4. The method of claim 3, wherein the starting position as the current position of the instrument is ascertained from the two-dimensional projection images of the at least one current projection geometry using the vascular model, using a three-dimensional image dataset, using a tracking system of the X-ray facility, or using any combination thereof.

5. The method of claim 1, wherein support points are defined at least at intersections of the vascular structure at which a plurality of vessels converge, are selected at least partially at a specific interval along at least one center line of a vessel of the path, or a combination thereof.

6. The method of claim 1, wherein a first term of the at least one first term is selected from the group comprising:
for support points at, in, or at and in a tolerance range around bifurcations of the vascular structure, a term for minimizing a deviation of a projection direction of the projection geometry from a cross product of a running direction of the vessels of a bifurcation at the bifurcation in accordance with the vascular model;
for support points that are not located at a bifurcation, are located outside of the tolerance range, or a combination thereof, a term that minimizes an optical foreshortening of at least the vessel in which the support point is located, of all vessels, or a combination thereof, visible in the projection geometry, of the path from the support point to the target position, is weighted less, in the vascular structure;
a term that promotes visibility of the instrument in the center of a projection image recorded with the projection geometry;
a term that maximizes a visible length of vessels along the path from the support point to the target position;
a term that minimizes a dose load for the examination object, at least one individual carrying out the treatment, an operator, or any combination thereof; and
a term that minimizes shadowing of at least part of the vascular structure, by other structures, in particular bones, of the examination object.

7. The method of claim 6, wherein the first term is the term that minimizes the shadowing of the instrument, the shadowing of the path from the support point to the target position, or a combination thereof.

8. The method of claim 6, wherein the other structures comprise bones of the examination object.

9. The method of claim 1, wherein a second term of the at least one second term is selected to be proportional to a number of changes in the projection geometry along the path, each change in the projection geometry is weighted with a magnitude of the respective change, or a combination thereof.

10. The method of claim 1, wherein for the second term, a change in the projection geometry is ascertained using an alteration in at least one positioning parameter of the support, from one support point to the next.

11. The method of claim 10, wherein the at least one positioning parameter of the support includes angulation angles of the support.

12. The method of claim 1, wherein for each projection geometry, at least one collimation parameter that describes a collimation of projection images to be recorded, a zoom parameter that describes a zoom of projection images to be recorded, or a combination thereof is also ascertained.

13. The method of claim 1, wherein at least one constraint is used as a constraint term of the target function, as one to be additionally checked, or a combination thereof.

14. The method of claim 1, wherein a current position of the instrument in the vascular structure is tracked, and
wherein on reaching a support point, at which a change in the projection geometry takes place in accordance with the optimized course, after confirmation by a user, the positioning facility is actuated by the positioning parameter set of the projection geometry to be newly ascertained.

15. The method of claim 14, wherein the confirmation by the user takes place together with activation of X-ray radiation, on operation of a foot pedal, or a combination thereof.

16. The method of claim 1, wherein on a deviation by a user from the projection geometries of the optimized course, a new ascertainment takes place in accordance with the optimization process, starting from the deviating projection geometry that has been adjusted.

17. An X-ray facility comprising:
a support on which an X-ray tube assembly and an X-ray detector are arranged opposite each another;
a positioning facility for moving at least the support in order to adjust a projection geometry; and
a control facility comprising:
a first interface for supplying a vascular model, three-dimensionally describing a course of a vessel of a vascular structure of an examination object, from which projection images are to be recorded with the X-ray facility, wherein a target position of an instrument that is movable in the vascular structure is marked in the vascular model;
a second interface for supplying a starting position of the instrument in the vascular structure of the vascular model;
a third interface for supplying a path in the vascular structure of the vascular model from the starting position of the instrument to the target position;
a defining unit for defining support points along the path; and
an ascertainment unit configured to:
ascertain an optimized course of projection geometries for the support points in an optimization process of a target function using the vascular model, wherein, apart from at least one first term that is based on the optimization of image contents for an observer in the case of the instrument situated at the respective support point, the target function also comprises at least one second term that minimizes a number of changes in the projection geometry along the path due to movement of the support; and
ascertain positioning parameters for actuating the positioning facility for each projection geometry.

18. The X-ray facility of claim 17, wherein the vascular model describes the course of the vessel by center lines.

19. In a non-transitory computer-readable storage medium that stores instructions executable by one or more processors to operate an X-ray facility with a support on which an X-ray tube assembly and an X-ray detector are arranged opposite each other, and a positioning facility for moving at least the support, such that a projection geometry is adjusted, the instructions comprising:
supplying a vascular model, three-dimensionally describing a course of the vessel, of a vascular structure of an examination object, from which projection images are to be recorded with the X-ray facility, wherein a target position of an instrument that is movable in the vascular structure is marked in the vascular model;
supplying a starting position of the instrument in the vascular structure of the vascular model;
supplying a path in the vascular structure of the vascular model from the starting position of the instrument to the target position;
defining support points along the path;
ascertaining an optimized course of projection geometries for the support points in an optimization process of a target function using the vascular model, wherein, apart from at least one first term that is based on the optimization of the image contents for an observer in the case of an instrument situated at the respective support point, the target function also comprises at least one second term that minimizes a number of changes in the projection geometry along the path due to movement of the support; and
ascertaining positioning parameters for actuating the positioning facility for each projection geometry.
